(19) United States
(12) Patent Application Publication
(10) Pub. No.: US 2023/0302174 A1
(43) Pub. Date: Sep. 28, 2023

(54) SELF-SANITIZING HANDLE

(71) Applicants: The University of British Columbia, Vancouver (CA); Acuva Technologies Inc., Burnaby (CA)

(72) Inventors: Fariborz TAGHIPOUR, Burnaby (CA); Mahyar Mohaghegh MONTAZERI, Burnaby (CA); Babak Adeli KOUDEHI, Vancouver (CA); Majid KESHAVARZFATHY, Burnaby (CA)

(21) Appl. No.: 18/128,189

(22) Filed: Mar. 29, 2023

Related U.S. Application Data
(63) Continuation of application No. PCT/CA2021/051364, filed on Sep. 29, 2021.
(60) Provisional application No. 63/085,959, filed on Sep. 30, 2020.

(51) Int. Cl. A61L 2/10; A61L 2/26; E05B 1/00

(52) U.S. Cl. CPC A61L 2/10; A61L 2/26; E05B 1/0069; A61L 2202/11

(57) ABSTRACT

A handle comprises a grip and an ultraviolet radiation emitter operable to emit ultraviolet radiation to disinfect the grip. The grip has a body extending in a longitudinal direction between a proximal end surface located at a proximal end of the grip and a distal end surface located at an opposing distal end of the grip. The ultraviolet radiation emitter is located at the proximal end of the grip and configured to emit ultraviolet radiation. In some embodiments, the ultraviolet radiation emitter is configured to direct ultraviolet radiation from the proximal end and in directions oriented toward the body.

### REFERENCE TO RELATED APPLICATION

This application is a continuation of Patent Cooperation Treaty (PCT) application No. PCT/CA2021/051364 having an international filing date of 29 Sep. 2021 which in turn claims priority from, and, for the purposes of the United States, the benefit of 35 USC 119 in connection with, U.S. application No. 63/085,959 filed 30 Sep. 2020. All of the applications referred to in this paragraph are hereby incorporated herein by reference.

### TECHNICAL FIELD

The present invention relates generally to sanitization of frequently touched surfaces, and in particular to self-sanitizing handles (e.g. door handles) which include one or more ultraviolet (UV) radiation emitters. The present invention may be embodied as a standalone module which may be retrofitted to existing frequently touched surfaces such as existing handles, as an integrated self-sanitizing handle which may be retrofitted onto existing objects (e.g. doors), or as an object's handle.

### BACKGROUND

Microbial pathogens (e.g. viruses, bacteria, etc.) can spread from person to person via contaminated frequently touched surfaces. That is, microbial pathogens can be introduced into a person's body through the person's hands when the person touches a contaminated surface and subsequently touches their nose and/or mouth. Some microbial pathogens can live on surfaces for several hours or even several days. Hence, frequently touched surfaces such as door handles pose a big risk for transmitting pathogens, especially in situations like a pandemic, and it is generally desirable to disinfect such surfaces frequently.

UV radiation is known to be effective in neutralizing germs such as bacteria, viruses and fungi by damaging the DNA of the germs such that they become incapable of reproducing. Accordingly, it is desirable to use UV radiation for disinfecting frequently touched surfaces. One problem is that most UV sterilizers must be manually operated (e.g. by a janitor or custodian). Since frequently touched surfaces like door handles require frequent disinfection, it is not practical to disinfect frequently touched surfaces manually using traditional UV sterilizers. Another problem is that most UV sterilizers are not designed to disinfect tubular shaped objects like door handles. That is, most UV sterilizers are not operable to deliver sufficient and/or consistent amounts of UV radiation onto the entire surface of tubular shaped objects like door handles.

There remains a need for UV sterilizers which can be retrofitted onto or otherwise integrated with frequently touched surfaces such as door handles. There remains a need for such sterilizers to be operable automatically. Preferably such sterilizers should be designed to deliver sufficient and/or consistent amounts of UV radiation onto the entire surface of the frequently touched object.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a handle comprising: a grip having a body extending in a longitudinal direction between a proximal end surface located at a proximal end of the grip and a distal end surface located at an opposing distal end of the grip; an ultraviolet radiation emitter located at the proximal end of the grip and configured to direct ultraviolet radiation from the proximal end and in directions oriented toward the body. The ultraviolet radiation emitter comprises: an ultraviolet radiation source operable to emit the ultraviolet radiation; and a housing disposed at least partially around the ultraviolet radiation source with the ultraviolet radiation source located at least partially in a cavity defined at least in part by the housing.

The housing may comprise a reflector having a reflective surface disposed at least partially around the ultraviolet radiation source.

The ultraviolet radiation source may be located at least partially in a reflector concavity defined at least in part by the surface of the reflector.

One aspect of the invention relates to a handle comprising a grip and an ultraviolet radiation emitter. The grip has a body extending in a longitudinal direction between a proximal end surface located at a proximal end of the grip and a distal end surface located at an opposing distal end of the grip. The ultraviolet radiation emitter is located at the proximal end of the grip and configured to direct ultraviolet radiation from the proximal end and in directions oriented toward the body. The ultraviolet radiation emitter comprises an ultraviolet radiation source operable to emit the ultraviolet radiation and a reflector. The reflector has a concave surface disposed around the ultraviolet radiation source.

In some embodiments, the ultraviolet radiation emitter is configured to direct ultraviolet radiation from the proximal end in directions oriented toward the body of the grip and having components oriented in the longitudinal direction and orthogonal to the longitudinal direction. In some embodiments, the concave surface is shaped for receiving the ultraviolet radiation from the ultraviolet radiation source and for directing the ultraviolet radiation received from the ultraviolet radiation source in the directions oriented toward the body of the grip and having components oriented in the longitudinal direction and orthogonal to the longitudinal direction.

In some embodiments, the handle further comprises an auxiliary reflector located at the proximal end surface of the grip. The auxiliary reflector is oriented to face toward the ultraviolet radiation source to reflect the ultraviolet radiation emitted by the ultraviolet radiation source toward the concave surface of the reflector. The auxiliary reflector may have a planar, concave, or convex reflective surface.

In some embodiments, the handle further comprises an end cover located at the distal end of the grip. The end cover may comprise a reflective surface configured to direct the ultraviolet radiation received from the ultraviolet radiation emitter from the distal end in the directions oriented toward the body and having components oriented in the longitudinal direction and orthogonal to the longitudinal direction. The end cover may have a planar or concave reflective surface. The end cover may span an area greater than the area of the distal end surface. The end cover may be attached to the distal end surface.

In some embodiments, the handle further comprises a second ultraviolet radiation emitter located at the distal end of the grip. The second ultraviolet radiation emitter is configured to direct ultraviolet radiation from the distal end and in directions oriented toward the body. The second ultraviolet radiation emitter may comprise a second ultraviolet radiation source operable to emit the ultraviolet radiation and a second reflector having a second concave surface disposed around the second ultraviolet radiation source. The second ultraviolet radiation emitter may be configured to direct ultraviolet radiation from the distal end in directions oriented toward the body and having components oriented in the longitudinal direction and orthogonal to the longitudinal direction. The second concave surface may be shaped for receiving the ultraviolet radiation from the second ultraviolet radiation source and for directing the ultraviolet radiation received from the second ultraviolet radiation source in the directions oriented toward the body and having components oriented in the longitudinal direction and orthogonal to the longitudinal direction.

In some embodiments, the handle further comprises a second auxiliary reflector located at the distal end surface of the grip. The second auxiliary reflector is oriented to face toward the ultraviolet radiation source of the second ultraviolet radiation emitter to reflect the ultraviolet radiation emitted by the ultraviolet radiation source toward the concave surface of the reflector of the second ultraviolet radiation emitter. The second auxiliary reflector may have a planar, concave, or convex reflective surface.

In some embodiments, the ultraviolet radiation emitter comprises first and second radiation sources operable to emit ultraviolet radiation toward the concave surface of the reflector. In some embodiments, the ultraviolet radiation source is attached to the proximal end surface and the ultraviolet radiation emitter comprises a second reflector having a convex surface located in a concavity of the reflector. In some embodiments, the ultraviolet radiation source and/or the ultraviolet radiation emitter comprises a principal optical axis which is aligned with a central axis of the grip.

In some embodiments, the handle comprises a base coupled to the grip at the proximal end of the grip. The base comprises attachment mechanisms for attachment onto a door. The base may be configured to cause the longitudinal direction to be parallelly aligned with a leaf of the door when the handle is attached to the door. Alternatively, the base may be configured to cause the longitudinal direction to be orthogonally aligned with a leaf of the door when the handle is attached to the door.

In some embodiments, the concave surface of the reflector comprises a first section having a first curvature and a corresponding first focal point and a second segment having a second curvature and a corresponding second focal point. The first segment may have a parabolic curvature and the second segment may have a hyperbolic curvature. The ultraviolet radiation source may be spaced apart from the first and second focal points.

Another aspect of the invention relates to a handle comprising a grip having a body made of a UV transparent material and an UV radiation source. The body extends between a proximal end surface located at a proximal end of the grip and a distal end surface located at an opposing distal end of the grip. The UV radiation source is operable to emit UV radiation. The UV radiation source is located at the proximal end of the grip and optically oriented to emit UV radiation through the proximal end surface and into an interior of the body of the grip. The body has a shape and an index of refraction selected to cause the UV radiation in the interior of the body to undergo total internal reflection at an interface between the body and air as the UV radiation travels in the interior of the body.

In some embodiments, contact residue on an external surface of the body causes radiation in the interior of the body to be transmitted to an outside of the body and into the contact residue because of the difference between the index of refraction of the contact residue and air. The contact residue may be residue from contact between human skin and the body. The contact residue may comprise microorganisms.

In some embodiments, the handle further comprises a second UV radiation source located at the distal end of the grip and optically oriented to emit UV radiation through the distal end surface and into the interior of the body. In other embodiments, the handle further comprising an end reflector located at the distal end of the grip and the end reflector is configured to direct the UV radiation received from the UV radiation source toward the proximal end of the grip.

Handles described herein may optionally comprise a sensor for detecting a presence of a user and/or a user's hand in contact with or in proximity to the handle and control logic configured to turn the UV radiation source ON or OFF based on output from the sensor. The sensor may comprise one or more of: a motion sensor for detecting motion associated with the presence of the user's hand and/or the presence of the user, a light detection and ranging (LiDAR) sensor, a heat sensor for detecting heat associated with the presence of the user's hand, and a proximity sensor for detecting the proximity of the user's hand.

Another aspect of the invention provides a method for disinfecting surfaces of a handle. The method comprises: providing a handle comprising a grip having a body extending in a longitudinal direction between a proximal end surface located at a proximal end of the grip and a distal end surface located at an opposing distal end of the grip; locating an ultraviolet radiation emitter comprising an ultraviolet radiation source at the proximal end of the grip and orienting the ultraviolet radiation emitter to direct ultraviolet radiation from the proximal end and in directions oriented toward the body; and disposing a concave reflector around the ultraviolet radiation source with the ultraviolet radiation source located at least partially in a cavity defined by the concave reflector.

The methods may comprise any of the features combinations of features and/or sub-combinations of features of any of the handles discussed above.

Another aspect of the invention a method for disinfecting surfaces of a handle. The method comprises: providing any of the handles described above; sensing at least one of: a toggle off condition comprising at least one of: a presence of a user in a vicinity of the handle; a hand of a user in proximity with the handle; and a hand of the user in contact with handle; and a toggle on condition comprising at least one of: an absence of a user in a vicinity of the handle; an absence of a hand of a user in proximity with the handle; and an absence of hand of the user in contact with handle; and at least one of: causing the UV radiation source to turn ON (start emitting radiation) for at least a period of time based on sensing a toggle on condition; and causing the UV radiation source to turn OFF (stop emitting radiation) based sensing a toggle off condition.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic side view of a handle according to an example embodiment of the invention. FIG. 1A is an exploded perspective view of an example implementation of the FIG. 1 handle. FIG. 1B is an exploded sectional view of the FIG. 1A handle. FIG. 1C is a side view of the FIG. 1A handle. FIG. 1D is a front perspective view of the FIG. 1A handle. FIG. 1E is a rear perspective view of the FIG. 1A handle. FIG. 1F is a side sectional view of the FIG. 1A handle. FIGS. 1G-H are perspective views of an example implementation of the FIG. 1 handle comprising a sensor.

FIGS. 2A-B illustrate exemplary radiation patterns emitted by a UV emitter of a type of the FIG. 1 handle.

FIG. 3 is a schematic side view of a door knob according to an example embodiment of the invention. FIG. 3A is an end view of an example implementation of the FIG. 3 door knob. FIG. 3B is a perspective view of the FIG. 3A door knob. FIG. 3C is a top view of the FIG. 3A door knob. FIG. 3D is side sectional view of the FIG. 3A door knob.

FIG. 4 is a schematic side view of a handle comprising two UV emitters according to an example embodiment of the invention. FIG. 4A is a top view of an example implementation of the FIG. 4 handle. FIG. 4B is side view of the FIG. 4A handle. FIG. 4C is a perspective view of the FIG. 4A door handle. FIG. 4D is an end view of the FIG. 4A handle.

FIGS. 5A-B depict exemplary variations of the FIG. 1 handle.

FIG. 6 is a schematic side view of a UV transparent handle according to an example embodiment of the invention. FIG. 6A is a perspective view of an example implementation of the FIG. 6 handle. FIG. 6B-6D show example variations of the FIG. 6 handle. FIG. 6E is a perspective view of an example implementation of the FIG. 6 handle comprising a sensor.

FIG. 7 is a schematic side view of a UV transparent handle comprising two UV emitters according to an example embodiment of the invention. FIG. 7A is a perspective view of an example implementation of the FIG. 7 handle.

FIGS. 8A-F illustrate exemplary radiation patterns emitted by a UV emitter of the FIG. 6 handle.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

In this description and the accompanying claims, a number of surfaces are described as being reflective surfaces. Such reflective surfaces are coated with or otherwise comprise a material (e.g. aluminum) that is reflective to radiation emitted by an emitter. Such reflective surfaces may be substantially specularly reflective at the wavelengths of the radiation sources used in their particular embodiment or application. In some non-limiting embodiments, the radiation sources described herein may be UV-LED sources in the UV-C range (e.g. wavelengths on the order of 220-290 nm) and such reflective surfaces may be substantially specularly reflective at these UV-C wavelengths. Such reflective surfaces may be coated with or otherwise covered with UV-transparent protective materials, UV-enhanced materials and/or biocompatible materials (e.g. materials that are safe to be in contact with drinking water). Such materials may protect the surface from oxidation, corrosion, etc. Unless the context dictates otherwise, references herein to such reflective surfaces should be understood to include reflective surfaces covered by any such protective, UV-transparent, UV-enhanced and/or biocompatible materials (e.g. silicone dioxide, magnesium fluoride, UV transparent polymers, similar materials, combinations of such materials and/or the like), where present.

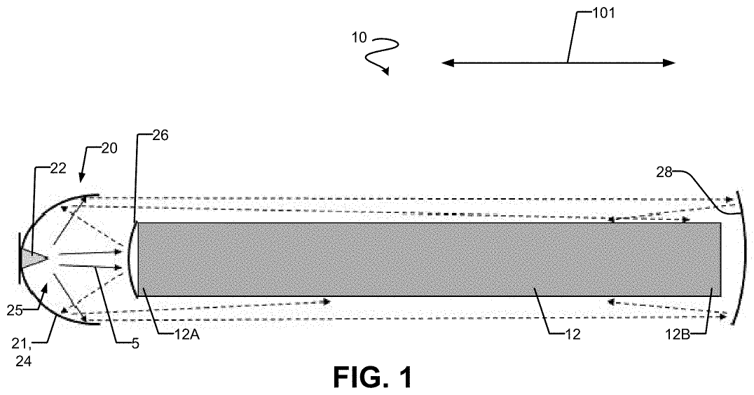

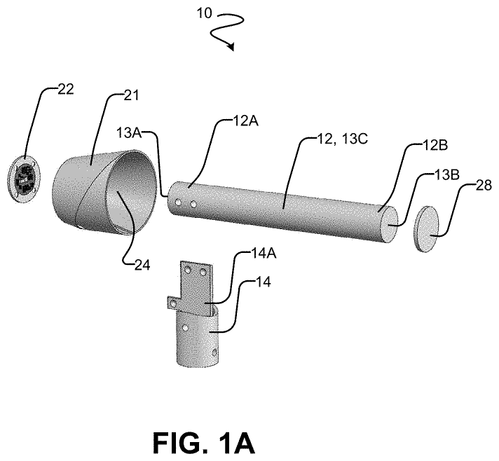

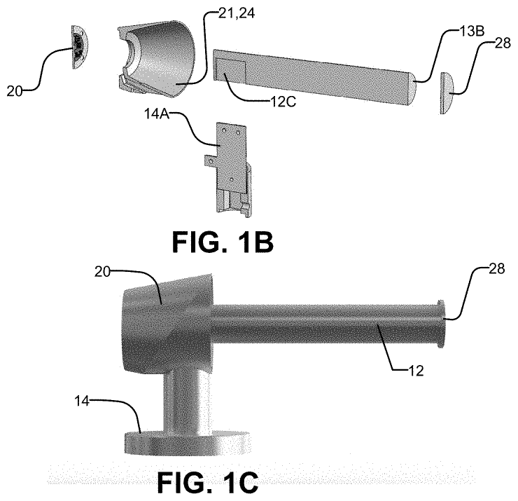

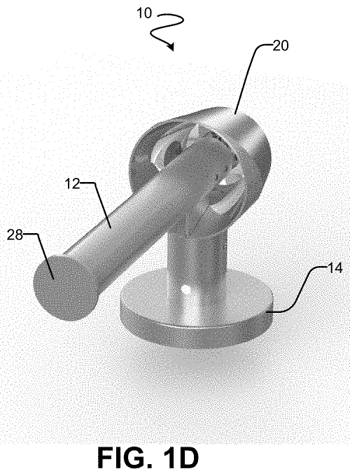

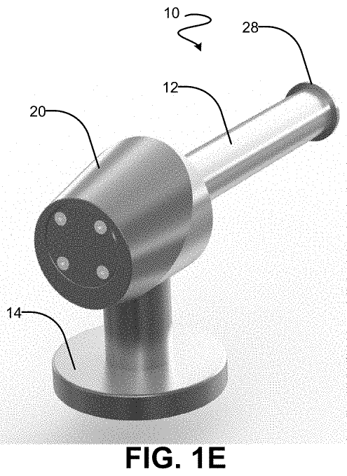

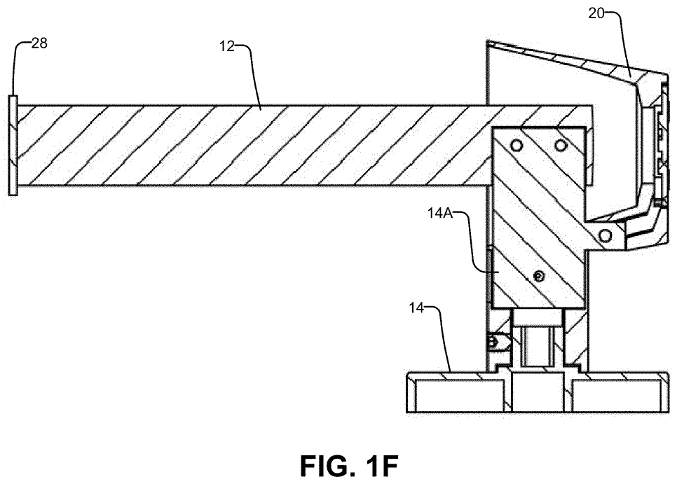

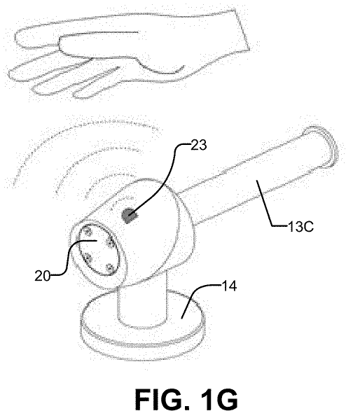

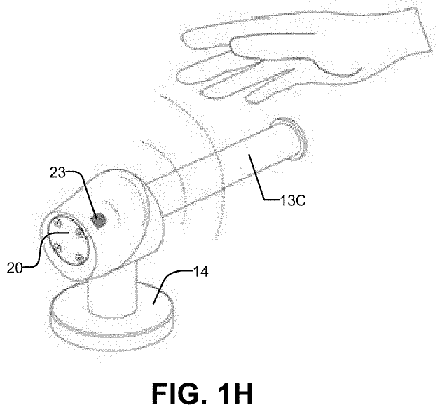

FIG. 1 is a schematic side view of a handle 10 according to an example embodiment of the invention. FIGS. 1A-F show various views of a potential implementation of the FIG. 1 handle 10. Handle 10 comprises an ultraviolet (UV) radiation emitter 20 located at a proximal end 12A of a grip 12. Grip 12 of the FIG. 1 handle 10 is elongated in a longitudinal direction 101 between proximal end 12A and an opposing distal end 12B. Grip 12 may comprise a body 13C extending (in longitudinal direction 101) between a proximal end surface 13A and a distal end surface 13B of grip 12 as shown in FIG. 1A. Although not necessary, grip 12 may be generally cylindrically shaped and may comprise end surfaces 13A, 13B and a generally cylindrically shaped body 13C. End surfaces 13A, 13B may have normal vectors which are predominantly parallel to longitudinal direction 101. Body 13C may have normal vectors which are predominantly orthogonal to longitudinal direction 101.

Grip 12 may be fabricated from any suitable materials, such as, without limitation, aluminum, stainless steel, or the like.

Grip 12 may be coupled to a base 14 at the proximal end 12A of grip 12. Grip 12 may be coupled to base 14 via any suitable attachment mechanism. For example, base 14 may comprise a flange 14A which may be inserted into a slot 12C located at the proximal end 12A of grip 12 as shown in FIG. 1B. Slot 12C may be provided at the surface of body 13C. As another non-limiting example, base 14 may be integrally formed with grip 12. Base 14 may be designed or otherwise adapted to attach onto objects such as doors, windows, drawers, taps, or any other object which may be moved or used by hand. Base 14 may comprise or otherwise receive, for example, suitable mechanical fasteners such as bolts, screws, or the like for attaching to such objects. When base 14 is attached to such objects, handle 10 may be used by a person to move or otherwise operate the object. For example, when base 14 is attached onto a door, the distal end 12B of grip 12 may in some cases be pivoted relative to base 14 to engage a latch of the door or to otherwise pull or push on the door.

In the illustrated embodiment of FIG. 1, UV radiation emitter 20 is located at the proximal end 12A of handle 10 as shown in FIG. 1. UV radiation emitter 20 may be coupled to base 14 as shown in FIG. 1F. In some embodiments, UV radiation emitter 20 and base 14 are integrally formed. In other embodiments, UV radiation emitter 20 is detachably coupled to base 14.

UV radiation emitter 20 comprises one or more UV radiation sources 22, such as one or more solid-state radiation emitters (e.g. UV-LEDs). UV radiation sources 22 may be operated manually (e.g. by a person pressing an ON and OFF button on handle 10) or automatically (e.g. by a microcontroller or by activation from a remote processor) to emit UV radiation 5. UV radiation sources 22 may be oriented to emit UV radiation 5 directly towards grip 12 as shown in FIG. 1. That is, where UV radiation source(s) 22 are UV-LEDs that have corresponding principal optical emission axes, such principal optical emission axes may be oriented in directions parallel to longitudinal direction 101. In some embodiments, the principal optical axes of UV radiation source(s) 22 may be aligned with a central axis (i.e. an axis located at the center of and normal to end surface 13A) of grip 12.

In some embodiments, UV radiation sources 22 are mounted on a printed circuit board (PCB), or the like, comprising auxiliary components (e.g. drivers, controllers, etc.) which may be configured to turn UV radiation sources 22 ON or OFF at specific times or to otherwise control the operation of radiation sources 22. Such PCBs may comprise thermally conductive PCBs such as metal-core PCBs or the like. In some embodiments, UV radiation emitter 20 comprises a sensor 23 (e.g. a motion sensor for detecting motion, a proximity sensor for detecting the presence of nearby objects, a light detection and ranging (LiDAR) sensor, etc.) and the auxiliary components of the PCB (e.g. a suitably configured controller or processor) may be configured to turn UV radiation sources 22 ON or OFF (e.g. for a period of time) based on a response of (e.g. a motion detected by) sensor 23. For example, UV radiation sources 22 may be ordinarily turned ON a controller may be configured to turn UV radiation sources 22 OFF upon sensor 23 detecting a person's hand approaching handle 10 as shown in FIG. 1G. Turning UV radiation sources 22 ON and OFF in this manner can advantageously prevent the person's hand from being directly exposed to UV radiation 5 when the person touches grip 12, while disinfecting grip 12 with UV radiation 5 whenever grip 12 is not being touched. As another example, UV radiation sources 22 may be ordinarily turned OFF and sensor 23 may be configured to detect motion which may be interpreted by a suitably configured controller to be a person's hand approaching or contacting grip 12, and upon the person's hand leaving grip 12 (e.g. an end of the detection motion or a period of time after the end of the detected motion), the controller may be configured to turn UV radiation sources 22 ON to direct UV radiation toward grip 12 and to thereby disinfect grip 12. The controller may be configured to turn ON UV radiation sources 22 a configurable period of time (e.g. 30 seconds). If the radiation is ON, controller may be configured to turn UV radiation sources 22 OFF upon sensor 23 detecting a person's hand re-approaching or contacting grip 12 to avoid irradiating the person's hand with UV radiation. Sensor 23 may be oriented to detect motion or the presence of an object (e.g. a person and/or a person's hand) near grip 12 (e.g. see FIG. 1H) and/or near UV radiation emitter 20 (e.g. see FIG. 1G). Sensor 23 may be located on the same PCB as UV radiation source(s) 22.

UV radiation sources 22 may be powered by any suitable power source. For example, UV radiation sources 22 may be powered by a rechargeable battery which may be integrated as part of UV radiation emitter 20. As another example, UV radiation sources 22 may be powered by an external battery, a building power supply, a building power source, a solar cell, or the like, through a removable or permanent power cord or through wireless power transfer (e.g. infrared light charging, magnetic induction charging, etc.).

UV radiation emitter 20 may comprise housing 21 disposed at least partial around UV radiation source 22 (e.g. in the form of a concave shape where UV radiation source 22 is located in the concavity). Housing 21 may comprises a reflective surface (or reflector) 24 having a concave reflective surface disposed around UV radiation source 22. Reflector 24 and other reflectors described herein (e.g. auxiliary reflector 26 and end cover 28) may be made of suitable thermally conductive material such as a metal, metal alloy, thermally conductive polymers, metal nitrites, metal nitrides, ceramics and/or the like.

In some embodiments, reflector 24 is partially conically or frustro-conically shaped. While described as conically shaped, reflector 24 does not need to be conically shaped or have a conically shaped reflective surface in the strict sense. For the purposes of facilitating the description, the term “cone” or “conical” is used herein for convenience and/or brevity to describe reflectors (e.g. reflector 24) which are shaped to define, at least in part, a reflector cone concavity (e.g. reflector cone concavity 25) having relatively large transverse cross-sectional areas (e.g. cross-sections on planes orthogonal to longitudinal direction 101 or having a normal vector parallel to longitudinal direction 101) at locations relatively closer to body 13C or grip 12 along longitudinal direction 101 and relatively small transverse cross-sectional areas at locations relatively further from body 13C or grip 12 along longitudinal direction 101. For example, reflector 24 may have the shape of a paraboloid, a hyperboloid, a semi-ellipsoid, a semi-sphere, or a combination thereof. In some embodiments, reflector 24 is truncated (e.g. frustro-conical) such that it does not have a singular apex (e.g. see FIG. 1B).

In some embodiments, UV radiation source 22 is located at a location offset from a focal point(s) of reflector 24 so that reflected rays are not collimated, but instead converge to irradiate the grip surface relatively uniformly. That is, UV radiation emitter 20 may be designed (e.g. reflector 24 may be shaped and/or located and/or UV radiation source 22 may be located at a location and/or oriented) or otherwise configured to direct UV radiation 5 toward the entire length of body 13C of handle 10 to ensure that parts of body 13C (i.e. at least those parts of body 13C away from the longitudinal ends and not covered by the reflector) receive a minimum threshold amount of UV irradiance (e.g. more than 5%, 10%, 15%, 20%, or 25% of the highest UV irradiance delivered to body 13C). In some embodiments, UV radiation emitter 20 may be designed (e.g. reflector 24 may be shaped and/or located and/or UV radiation source 22 may be located at a location and/or oriented) or otherwise configured to direct UV radiation 5 toward the entire length of body 13C of handle 10 to ensure that the variation of UV irradiance delivered to parts of body 13C (i.e. at least those parts of body 13C away from the longitudinal ends and not covered by the reflector) receive radiation that is uniform (e.g. within 20%, 25% or 30% irradiance variation between the maximally irradiated parts of the surface and the minimally irradiated parts of the surface).

One aspect of the invention provides handles 10 which comprise a UV radiation emitter 20 that is designed or otherwise configured to direct UV radiation 5 toward the body 13C of grip 12 to disinfect grip 12 when it is not in use (e.g. when it is not being touched by a person). Such UV radiation emitters 20 are designed or otherwise configured to direct UV radiation 5 from the proximal end 12A of the grip 12 (e.g. the end that is coupled to the object) toward and across the body 13C of grip 12. In some embodiments, UV radiation emitter 20 is configured to direct UV radiation 5 toward the body 13C of grip 12 in directions oriented toward the body and having components oriented in the longitudinal direction and orthogonal to the longitudinal direction. That is, UV radiation emitter 20 may be configured to emit UV radiation 5 which is not collimated, but which instead converges toward body 13C. UV radiation emitter 20 may be configured to deliver sufficient amounts of UV radiation 5 to disinfect grip 12 while keeping the amount of UV radiation 5 that is directed towards the environment below an acceptably low threshold. Accordingly, such UV radiation emitters 20 typically comprise a reflector 24 having a concave surface shaped to focus, converge, collimate and/or otherwise direct UV radiation 5 emitted by UV radiation source 22 toward and across the body 13C of grip 12.

UV radiation emitter 20 described herein may optionally comprise an auxiliary reflector 26 located at the proximal end 12A of grip 12 (i.e. the end where UV radiation emitter 20 is located). For example, UV radiation emitter 20 may comprise an auxiliary reflector 26 secured against or otherwise located at the proximal end surface 13A of grip 12 as shown in FIG. 1 (e.g. proximal end surface 13A may comprise a UV reflective surface 26). In the example embodiment shown in FIG. 1, auxiliary reflector 26 is oriented to face toward UV radiation source 22. Auxiliary reflector 26 may be shaped to reflect at least parts of UV radiation 5 emitted by UV radiation source 22 back towards reflector 24. Auxiliary reflector 26 may, for example, be flat or concave-shaped or, in the case of the FIG. 1 embodiment, convex-shaped. In some embodiments, auxiliary reflector 26 is a convex hyperbolic reflector.

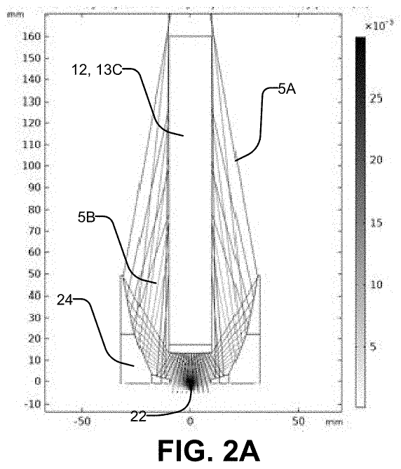

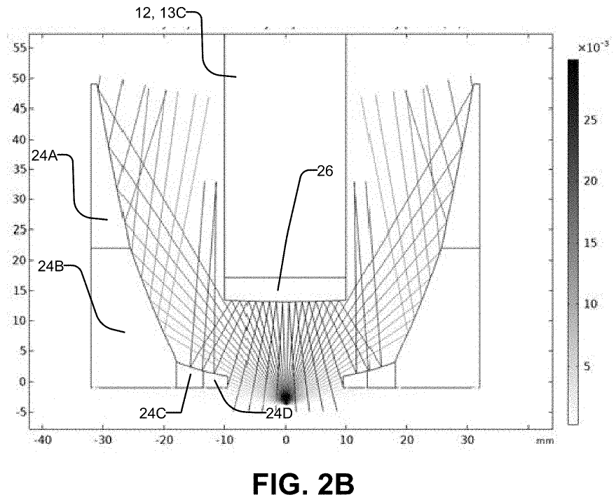

In some embodiments, auxiliary reflector 26 is shaped to reflect UV radiation 5 emitted by UV radiation source 22 to sections or segments of reflector 24 that are closer to the apex or to a notional apex of reflector 24 (i.e. portions that are closer to UV radiation source 22) as shown in FIGS. 2A-2B. In some embodiments, reflector 24 and/or auxiliary reflector 26 are shaped to direct UV radiation 5 emitted by UV radiation source 22 toward and across the entire body 13C of grip 12. That is, reflector 24 and/or auxiliary reflector 26 may be shaped to direct UV radiation 5 towards grip 12 in directions oriented toward the body and having components oriented in the longitudinal direction 101 and orthogonal to the longitudinal direction. In some embodiments, UV radiation emitter 20 directs UV radiation 5 towards body 13C of grip 12 and across the entire length of grip 12 to disinfect the entire body 13C of grip 12 (e.g. see FIG. 2A).

As depicted in FIGS. 2A-2B, UV radiation emitter 20 may direct both primary first order UV radiation 5A (i.e. UV radiation 5 which has been reflected once) and secondary second order UV radiation 5B (i.e. UV radiation 5 which has been reflected twice) toward the body 13C of grip 12. In some embodiments, reflector 24 is shaped to reflect first order UV radiation 5A toward locations on grip 12 that are relatively distal from UV radiation source 22 (i.e. locations that are closer to distal end 12B). In some embodiments, reflector 24 and/or auxiliary reflector 26 are shaped to reflect second order UV radiation 5B toward locations on grip 12 that are relatively proximate to UV radiation source 22 (i.e. locations that are closer to proximal end 12A).

Reflector 24 and/or auxiliary reflector 26 may be shaped to direct UV radiation 5 toward body 13C of grip 12 from different angles. That is, reflector 24 and/or auxiliary reflector 26 may be shaped to direct UV radiation 5 toward body 13C in a manner which causes different sections of grip 12 (e.g. different sections across longitudinal direction 101) to receive UV radiation 5 from different angles. This configuration can, in some cases, help UV radiation emitter 20 deliver sufficient amounts of UV radiation 5 and/or relatively uniform UV radiation 5 across the body 13C of grip 12 to increase energy efficiency.

In the example illustrated in FIG. 2B, reflector 24 comprises a first section 24A which is located most distal from UV radiation source 22 (compared to the other sections), a second section 24B which is located more proximate to UV radiation source 22 compared to first section 24A but more distal from UV radiation source 22 compared to third section 24C and fourth section 24D, a third section 24C which is located more proximate to UV radiation source 22 compared first section 24A and second section 24B but more distal from UV radiation source 22 compared to fourth section 24D, and a fourth section 24D which is located most proximate to UV radiation source 22 (compared to the other sections). Each of the sections 24A, 24B, 24C, 24D is shaped differently (e.g. has different curvatures) to direct UV radiation 5 toward body 13C at different impingement angles. For the purposes of facilitating the description, the term “impingement angle” is used herein to refer to the angle between body 13C and the rays of UV radiation 5 impinging on body 13C (e.g. the angle between the rays of UV radiation 5 and longitudinal axis 101). Each of the sections 24A, 24B, 24C, 24D may have different focal points. Reflector 24 may comprise any number (e.g. 2, 3, 4, 5, 6, etc.) of such sections. In some embodiments, reflector 24 comprises some sections which exhibit parabolic curvature and some sections which exhibit hyperbolic curvature. In some embodiments, UV radiation source 22 is located at a location offset from a focal point of reflector section 24A, 24B, 24C, 24D so that reflected rays are not collimated, but instead converge to irradiate the grip surface relatively uniformly.

In the example illustrated in FIG. 2B, first section 24A is shaped to reflect first order UV radiation 5A emitted by UV radiation source 22 at relatively high impingement angles compared to the other sections 24B, 24C, 24D, second section 24B is shaped to reflect first order UV radiation 5A at impingement angles which are lower compared to the impingement angle of first section 24A but higher compared to the impingement angles of third section 24C and fourth section 24D, third section 24C is shaped to reflect second order UV radiation 5B emitted by UV radiation source 22 and reflected by auxiliary reflector 26 at impingement angles which are lower compared to the impingement angle of first section 24A and second section 24B but higher compared to the impingement angle of fourth section 24D, and fourth section 24D is shaped to reflect second order UV radiation 5B at impingement angles which are relatively low compared to the impingement angles of first section 24A, second section 24B and third section 24C. Advantageously, this design may in some cases help UV radiation emitter 20 direct UV radiation 5 toward and across the entire body 13C of grip 12 as shown in FIG. 2A.

In some embodiments (as shown in FIGS. 1 and 1A), handle 10 comprises an end cover 28 located at the distal end 12B of grip 12. End cover 28 faces toward UV radiation emitter 20. End cover 28 typically spans or otherwise covers a larger cross-sectional area than the cross sectional area of grip 12 (i.e. the cross sectional area of body 13C). For clarity, the cross-sectional area of grip 12 refers to a cross-sectional area having a normal to the cross-sectional plane which is parallel to longitudinal direction 101.

End cover 28 may be fabricated from a non-UV transparent material. End cover 28 may be provided to block UV radiation 5 from leaking from the vicinity of handle 10. End cover 28 may be made of a UV reflective material or otherwise comprise a UV reflective surface in some embodiments.

End cover 28 may be attached to distal end surface 13B as shown in FIG. 1C. Where end cover 28 is attached to distal end surface 13B, end cover 28 does not need to have a surface which is entirely reflective. Instead, end cover 28 may have an annular shaped reflective surface that forms a rim around its central non-reflective surface (i.e. the central non-reflective surface which is attached to distal end surface 13B). Advantageously, such design can reduce costs associated with the amount of reflective materials required to make end cover 28.

In some embodiments, end cover 28 comprises a concave reflective surface (or comprises a concave reflective rim) shaped to direct UV radiation 5 received from UV radiation emitter 20 back toward body 13C as shown in FIG. 1. End cover 28 may be designed to reflect first order UV radiation 5A and/or second order UV radiation 5B emitted by UV radiation emitter 20 toward the more distal portions of body 13C (i.e. portions of body 13C that are located at or near distal end 12B). Advantageously, end cover 28 can help reduce the amount of UV radiation 5 that escapes into the environment, thereby permitting more energy efficient operation and reducing stray radiation.

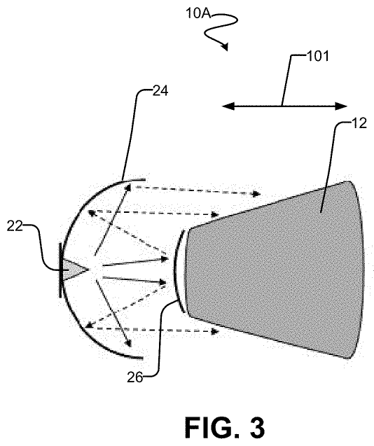

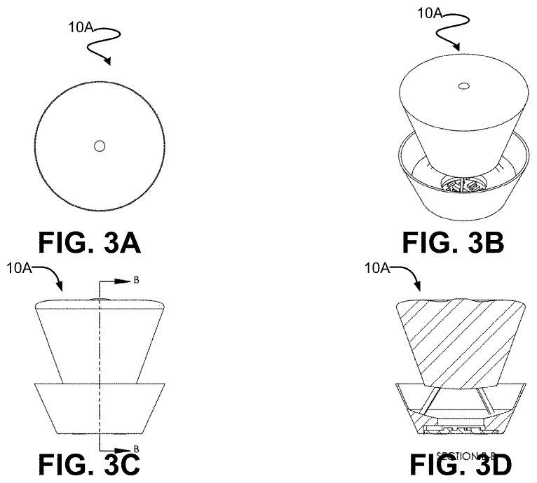

A wide range of variations are possible within the scope of the present invention. These variations and/or aspects thereof may be applied to all of the embodiments described herein. Non-limiting examples of such variations are illustrated in the various embodiments shown in FIGS. 3-5 of the drawings and described in more detail below.

FIG. 3 is a schematic side view of a handle 10A according to an example embodiment of the invention. FIGS. 3A-D show various views of a potential implementation of the handle 10A shown in FIG. 3. Handle 10A comprises an ultraviolet (UV) radiation emitter 20 coupled to a grip 12 that has the shape of a knob. Although not necessary, handle 10A is typically attached onto a door. When handle 10A is attached onto a door, grip 12 may in some cases be rotated (e.g. about longitudinal axis 101) to engage a latch of the door. When handle 10A is attached onto a door, longitudinal direction 101 typically extends in a direction normal to the surface of the door (instead of in a direction which is parallel the surface of the door, which is typically the case in the FIG. 1 embodiment).

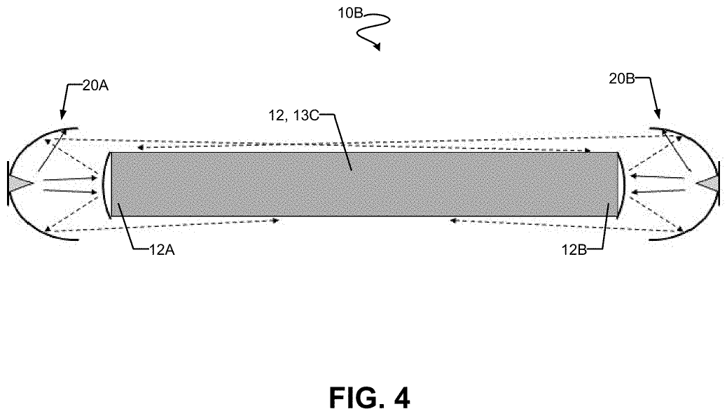

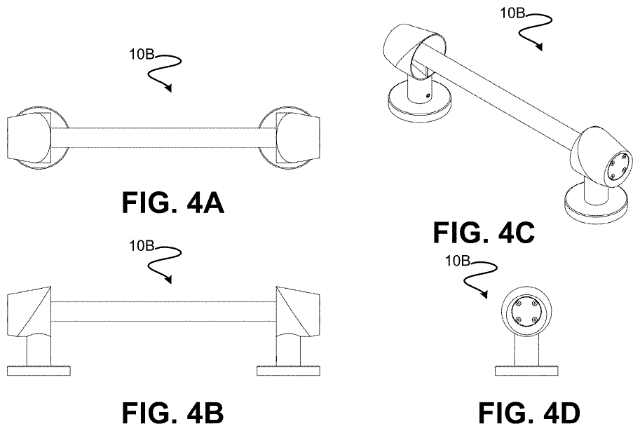

FIG. 4 is a schematic side view of a handle 10B according to an example embodiment of the invention. FIGS. 4A-D show various views of a potential implementation of the handle 10B shown in FIG. 4. Handle 10B comprises a first ultraviolet (UV) radiation emitter 20A coupled to a first end 12A of grip 12 and a second UV radiation emitter 20B coupled to a second end 12B of grip 12. As depicted in FIGS. 4A-4D, grip 12 may be coupled to a first base 14A at the first end 12A of grip 12 and a second base 14B at the second end 12B of grip 12.

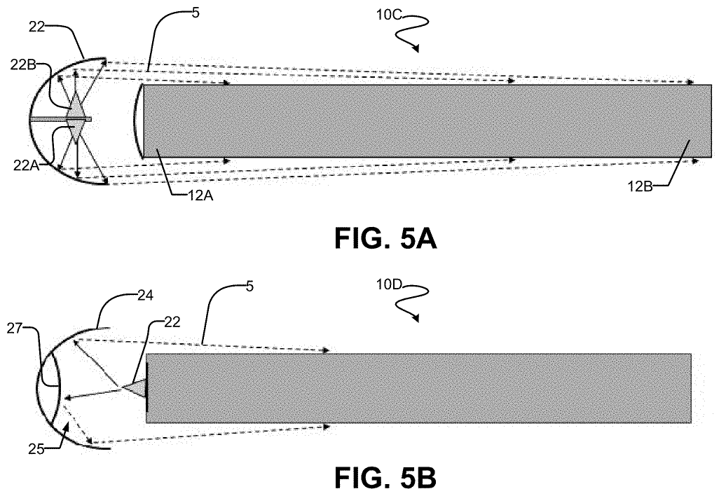

FIG. 5A is a schematic side view of a handle 10C according to an example embodiment of the invention. Handle 10C comprises a UV radiation emitter 20 that has two UV radiation sources 22A, 22B. UV radiation sources 22A, 22B are oriented to face toward reflector 24 instead of toward an end surface 13 of grip 12. As depicted in FIG. 5A, Handle 10C may comprise first and second UV radiation sources 22A, 22B that are oriented to align their respective principal optical axes in directions that are perpendicular to the direction to longitudinal direction 101. In some embodiments, first and second UV radiation sources 22A, 22B are oriented to align their respective principal optical axes in directions that antiparallel to each other.

FIG. 5B is a schematic side view of a handle 10D according to an example embodiment of the invention. Handle 10D comprises a UV radiation emitter 20 that has a UV radiation source 22 attached to an end surface of 13 of grip 12. UV radiation source 22 is oriented to face toward reflector 24. In the example embodiment shown in FIG. 5B, UV radiation source 22 is oriented to align its principal optical axes in a direction that is parallel to longitudinal direction 101. As depicted in FIG. 5B, reflector 24 may comprise a convex-shaped reflector 27 provided in concavity 25 and oriented to reflect UV radiation 5 emitted by UV radiation source 22 towards reflector 24.

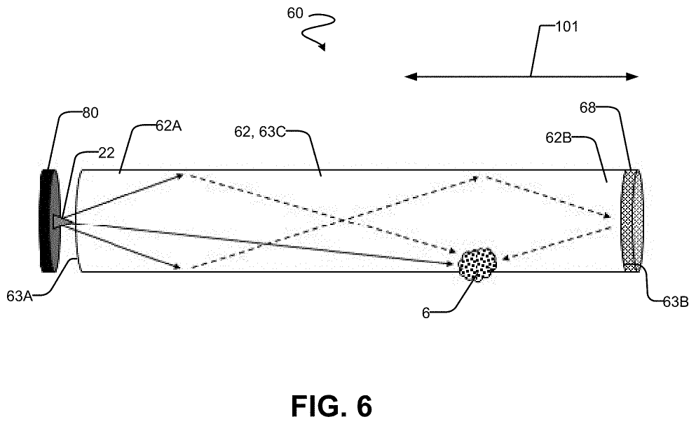

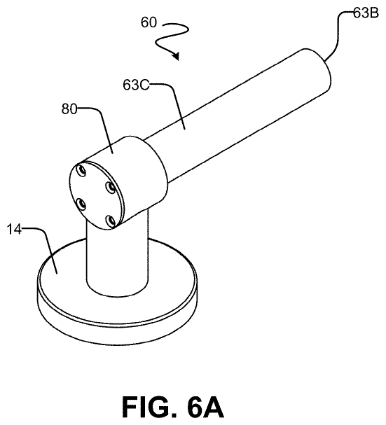

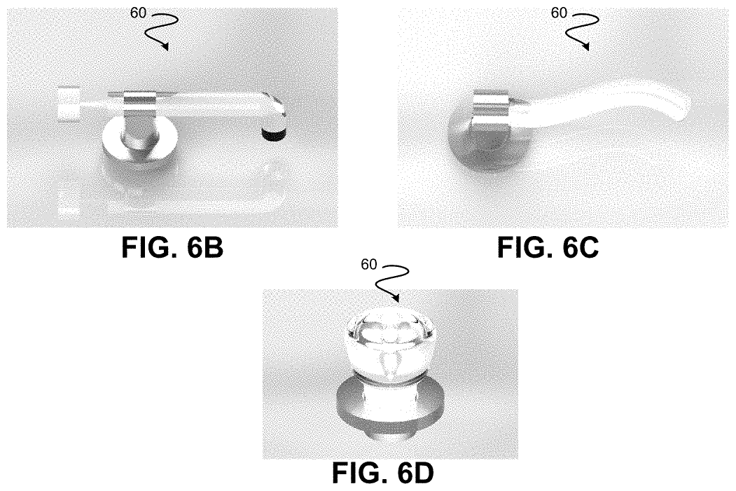

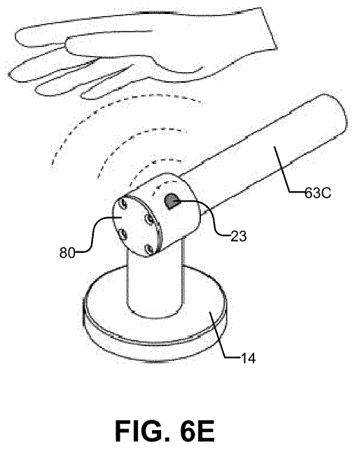

FIG. 6 is a schematic side view of a UV transparent handle 60 according to an example embodiment of the invention. FIG. 6A is a perspective view of an example implementation of UV transparent handle 60. Handle 60 comprises an ultraviolet (UV) radiation source 22 located at a proximal end 62A of a grip 62. Grip 62 is elongated in longitudinal direction 101 and extends between a proximal end 62A and a distal end 62B. Grip 62 comprises a body 63C located between a proximal end surface 63A and a distal end surface 63B as shown in FIG. 6A. Grip 62 and/or UV radiation emitter 80 may be coupled to a base 14 as shown in FIG. 6A.

Grip 62 is made of suitable UV transparent materials such as quartz, fused silica, etc. Handle 60 comprises a UV radiation source 22 operable to emit UV radiation 5. UV radiation source 22 is located at proximal end 62A of grip 62 and optically oriented to emit UV radiation 5 through the proximal end surface 63A of grip 62 and into an interior of the body 63C of the grip 62. Unless context dictates otherwise, the term “optically oriented” (as used herein) should be interpreted to imply that handle 60 may include optical elements (e.g. lenses, reflectors, waveguides, etc.) located in the optical path between UV radiation source 22 and grip 62 to direct UV radiation 5 into the interior of the body 63C of grip 62.

In some embodiments, UV radiation source 22 is provided as part of a UV radiation emitter 80 coupled to grip 62 at the proximal end 62A of grip 62. UV radiation emitter 80 may be secured against the proximal end surface 63A of grip 62 as shown in FIG. 6. In some embodiments, the segment of body 63C (i.e. segments close to proximal end 62A) which receives UV radiation emitter 80 comprises or is otherwise coated with a UV reflective material to reduce UV adsorption to UV radiation emitter 80 to thereby increase energy efficiency.

As described above, UV radiation sources 22 are optically oriented to face toward grip 12. In some embodiments, UV radiation source 22 is optically oriented to align its principal optical axis in a direction which is parallel to longitudinal direction 101. In some embodiments, UV radiation emitter 80 comprises a UV radiation source 22 having a principal optical axis which is aligned with a central axis of grip 62. In some embodiments, UV radiation source 22 has a principal optical axis which is optically aligned with the normal axis of proximal end surface 63A.

Preferably grip 62 is fabricated from a UV transparent material having a shape and a refractive index that is selected (e.g. based on the angle of incidence of UV radiation sources 22) to cause the UV radiation 5 in the interior of body 63C to undergo total internal reflection (TIR) at an interface between body 63C and air as the UV radiation 5 travels in the interior of body 63C. That is, grip 62 should preferably be made of a UV transparent material having a refractive index which causes UV radiation 5 emitted by UV radiation sources 22 to undergo TIR at the interface between the UV transparent material and air across the angles of incidence of UV radiation sources 22. In some embodiments, grip 62 is made of a UV transparent material having a refractive index which is above about 1.414. This helps ensure that UV radiation 5 traveling through grip 62 undergoes TIR at all (or almost all) incident angles when grip 62 is in contact with air. In other embodiments, grip 62 is made of a UV transparent material having a refractive index which is between 1 to 1.414. Advantageously, the TIR phenomena helps prevent UV radiation 5 from escaping the interior of body 63C when grip 62 is clean to avoid inadvertently delivering harmful radiation to, for example, a person's eyes. Since UV radiation source 22 can be turned ON constantly without risk of releasing UV radiation 5 to the environment, grip 60 may be operated (e.g. powered by a battery) continuously whenever grip 60 is not being touched (e.g. by a person's hand). One or more sensors and suitably configured controllers can be used (in a manner similar to that discussed above in relation to the control of the FIG. 1 handle 10) to control the ON/OFF state of radiation source 22.

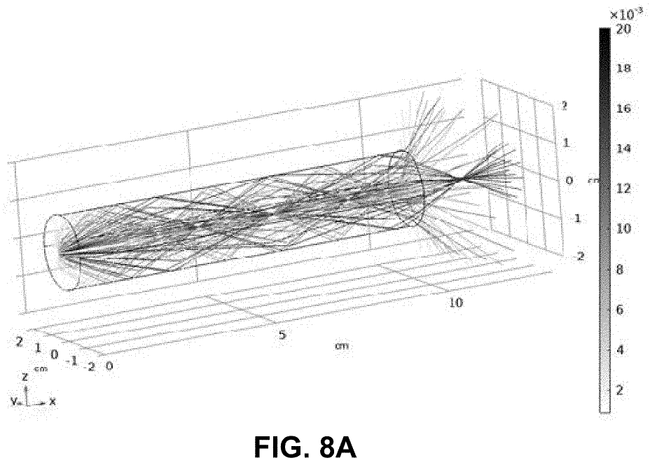

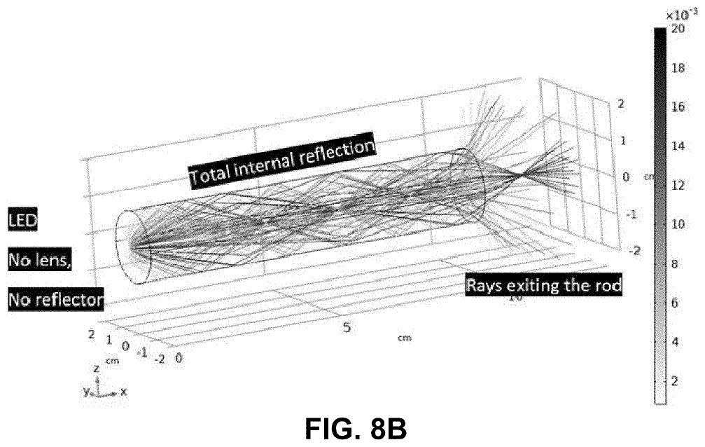

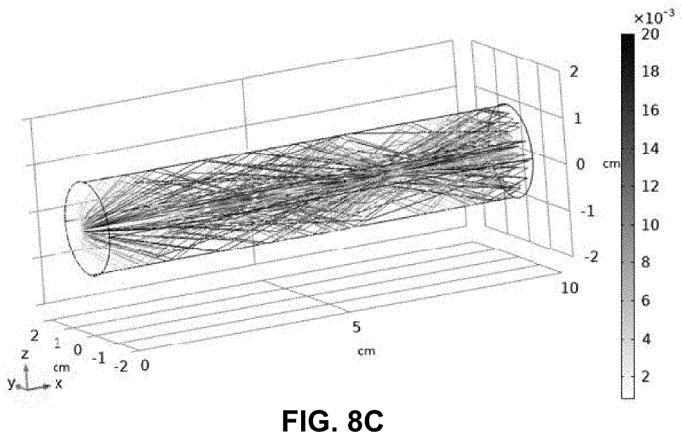

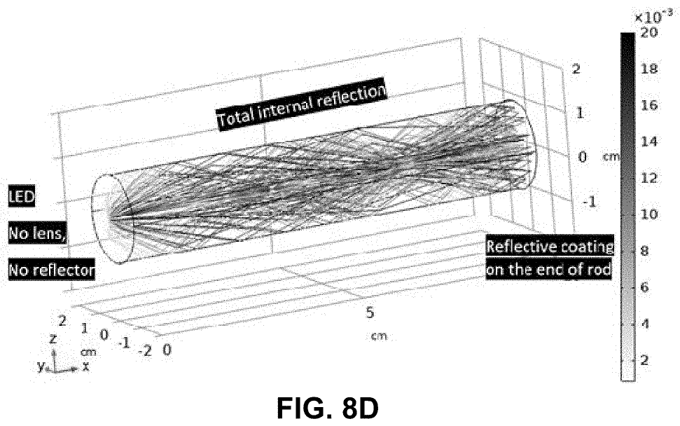

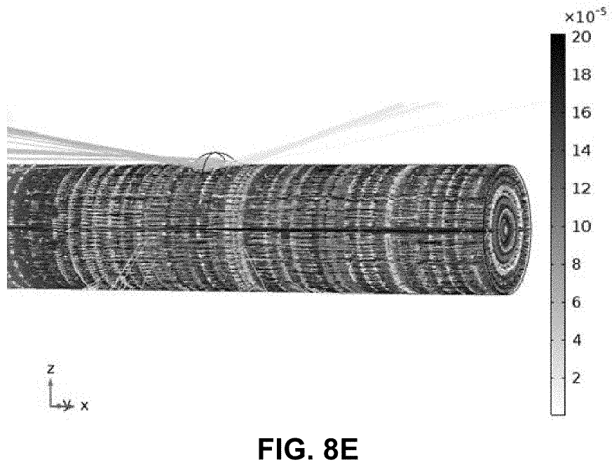

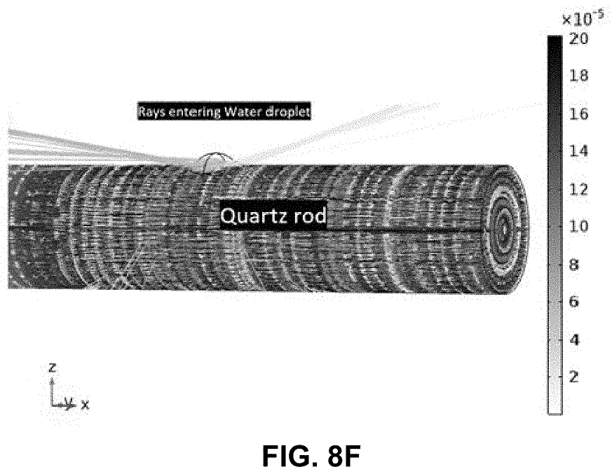

When something (other than air) contacts grip 62, such contact may leave contact residue 6. When contact residue 6 other than air (e.g. water droplets containing microbial pathogens, viruses, bacteria, etc.) is in contact with grip 62, the TIR phenomenon is impaired in the contact region (i.e. due to the presence of the contact residue 6, which has a refractive index different than that of air, at the interface between grip 62 and air). Consequently, UV radiation 5 in the interior of body 63C will exit body 63C when contact residue 6 accumulates on the surface of grip 62 to irradiate the residue which is in contact with grip 62 and to disinfect grip 62 and any contaminants which may be present in contact residue 6 (e.g. see FIG. 8E-F).

In some embodiments, handle 60 comprises an end reflector 68 located at the distal end 62B of grip 62. End reflector 68 faces toward UV radiation emitter 20. End reflector 68 typically spans or otherwise covers the entire cross-sectional area of grip 62 (i.e. the cross sectional area of body 63C). In some embodiments, end reflector 68 is attached to distal end surface 63B. In other embodiments, end reflector 68 is a reflective coating which may be applied directly at the distal end surface 63B part of grip 62.

Providing end reflector 68 at the distal end 62B of grip 62 can advantageously help prevent UV radiation 5 emitted by UV radiation source 22 from exiting grip 62 at end surface 63B. FIGS. 8A-E depict the radiation profile within a grip 62 comprising end reflector 68. As depicted in FIGS. 8A-E, UV radiation 5 emitted by UV radiation source 22 is contained within grip 62 due to TIR and the reflectivity of end reflector 68 and does not exit into the environment at end surface 63B. UV radiation 5 emitter by UV radiation source 22 may undergo TIR several (e.g. 2, 3, 4, 5, 6, 7 etc.) times within grip 62 as it travels from proximal end 62A to distal end 62B.

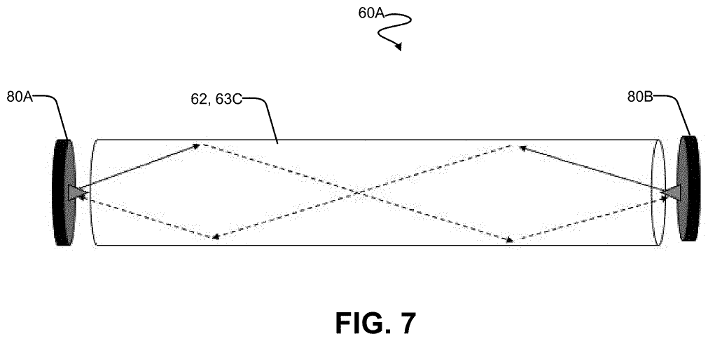

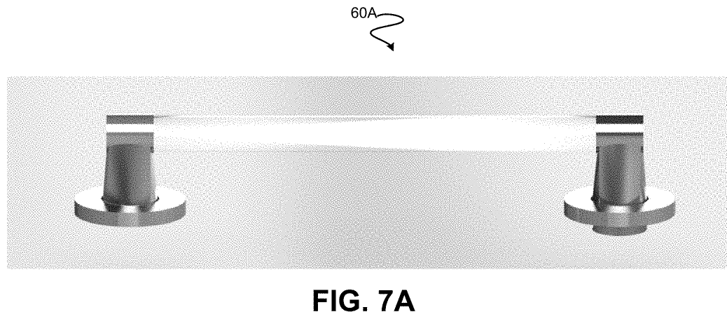

FIG. 7 is a schematic side view of a handle 60A according to an example embodiment of the invention. FIG. 7A shows a perspective view of a potential implementation of the handle 60A shown in FIG. 7. Handle 60A comprises a first UV radiation emitter 80A coupled to a first end 62A of grip 62 and a second UV radiation emitter 80B coupled to a second end 62B of grip 62. First and second UV radiation emitters 80 are provided at the end surfaces 63 of grip 62 to emit UV radiation 5 through the body 63C of grip 62 and toward the surface of grip 62 as depicted in FIG. 7.

The scope of the present invention includes a variety of possible supplementary designs to handles 10, 60 and/or other aspects of handles 10, 60. Where suitable, these variations may be applied to any of the handle embodiments described herein and include, without limitation the following:
handles 10, 60 may include one or more sensors (e.g. sensor 23) configured to detect a person's hands or other objects. Such sensors may include any suitable type(s) of motion sensors, radiation sensors, proximity sensors, electric field sensors, heat sensors and/or the like. The response of the sensor 23 can be employed for controlling the operation of UV radiation source 22. For example, sensor 23 may be utilized to control ON and/or OFF periods of UV radiation source 22 and its intensity. Supportive electronics may be included in the handle 10, 60 to support such features;
the power associated with operating the handles 10, 60 may be supplied from various sources such as batteries, solar power, wall plugs and/or the like;
handles 10, 60 may include additional optical elements such as lenses or additional reflectors (not shown in the illustrated embodiments) which may be configured to guide UV radiation 5 emitted by UV radiation source 22 toward grip 12, 62;
UV radiation emitter 20, 80 and/or UV radiation source 22 may be located at a wide range of distances away from proximal end surface 13A, 63A; UV radiation emitter 20, 80 may comprise a UV transparent window (e.g. a quartz window) provided in front of UV radiation source 22 and/or reflector 24 to allow transmission of UV radiation 5 while protecting UV radiation emitter 20, 80 from the environment (e.g. preventing air and/or water from contacting UV radiation source 22 and/or reflector 24);
handles 10, 60 may include a UV sensor located at the distal end 12B, 62B of the grip 12, 62 to monitor the intensity of UV radiation 5 and/or the performance of handle 10, 60 (e.g. dust on any reflector or dirt on any UV transparent material may negatively impact performance) and may issue suitable warnings if the intensity of radiation detected falls below an acceptable threshold.

Handles 10, 60 may in some cases be embodied as a kit. That is, handles 10, 60 may be embodied as a collection of components that can be delivered to and/or assembled at a desirable location to form all or parts of handles 10, 60. Such a kit typically comprises one or a combination of components which form the handles 10, 60 described herein. Examples of such components include but are not limited to: grip 12, 62, UV radiation emitter 20, 80, base 14, etc.

The invention includes a number of non-limiting aspects. Non-limiting aspects of the invention include the following.
1. A handle comprising:
   a grip having a body extending in a longitudinal direction between a proximal end surface located at a proximal end of the grip and a distal end surface located at an opposing distal end of the grip;
   an ultraviolet radiation emitter located at the proximal end of the grip and configured to direct ultraviolet radiation from the proximal end and in directions oriented toward the body, the ultraviolet radiation emitter comprising:
      an ultraviolet radiation source operable to emit the ultraviolet radiation; and
      a housing disposed at least partially around the ultraviolet radiation source with the ultraviolet radiation source located at least partially in a cavity defined at least in part by the concave housing.
2. The handle of aspect 1 wherein the housing comprises a reflector having a reflective surface disposed at least partially around the ultraviolet radiation source.
3. The handle of aspect 2 wherein the ultraviolet radiation source is located at least partially in a reflector concavity defined at least in part by the surface of the reflector.
4. The handle of any one of aspects 2 to 3 or any other aspect herein wherein the ultraviolet radiation emitter is configured to direct ultraviolet radiation from the proximal end in directions oriented toward the body and having components oriented in the longitudinal direction and orthogonal to the longitudinal direction.
5. The handle of aspect 4 or any other aspect herein wherein the concave surface is shaped for receiving the ultraviolet radiation from the ultraviolet radiation source and for directing the ultraviolet radiation received from the ultraviolet radiation source in the directions oriented toward the body and having components oriented in the longitudinal direction and orthogonal to the longitudinal direction.
6. The handle of any one of aspects 2 to 5 or any other aspect herein further comprising an auxiliary reflector located at the proximal end surface of the grip, the auxiliary reflector oriented to face toward the ultraviolet radiation source to reflect the ultraviolet radiation emitted by the ultraviolet radiation source toward the concave surface of the reflector.
7. The handle of aspect 6 or any other aspect herein wherein the auxiliary reflector has a planar, concave, or convex reflective surface.
8. The handle of any one of aspects 2 to 7 or any other aspect herein further comprising an end cover located at the distal end of the grip.
9. The handle of aspect 8 or any other aspect herein wherein the end cover comprises a reflective surface configured to direct the ultraviolet radiation received from the ultraviolet radiation emitter from the distal end in the directions oriented toward the body and having components oriented in the longitudinal direction and orthogonal to the longitudinal direction.
10. The handle of aspect 9 or any other aspect herein wherein the end cover has a planar or concave reflective surface.
11. The handle of any one of aspects 8 to 10 or any other aspect herein wherein the end cover spans an area greater than the area of the distal end surface and wherein the end cover is attached to the distal end surface.
12. The handle of any one of aspects 2 to 7 or any other aspect herein further comprising a second ultraviolet radiation emitter located at the distal end of the grip and configured to direct ultraviolet radiation from the distal end and in directions oriented toward the body.
13. The handle of aspect 12 or any other aspect herein wherein the second ultraviolet radiation emitter comprises:
   a second ultraviolet radiation source operable to emit the ultraviolet radiation; and
   a second reflector having a second concave surface disposed around the second ultraviolet radiation source.
14. The handle of aspect 13 or any other aspect herein wherein the second ultraviolet radiation emitter is configured to direct ultraviolet radiation from the distal end in directions oriented toward the body and having components oriented in the longitudinal direction and orthogonal to the longitudinal direction.
15. The handle of aspect 14 or any other aspect herein wherein the second concave surface is shaped for receiving the ultraviolet radiation from the second ultraviolet radiation source and for directing the ultraviolet radiation received from the second ultraviolet radiation source in the directions oriented toward the body and having components oriented in the longitudinal direction and orthogonal to the longitudinal direction.
16. The handle of any one of aspects 13 to 15 or any other aspect herein further comprising a second auxiliary reflector located at the distal end surface of the grip, the second auxiliary reflector oriented to face toward the ultraviolet radiation source of the second ultraviolet radiation emitter to reflect the ultraviolet radiation emitted by the ultraviolet radiation source toward the concave surface of the reflector of the second ultraviolet radiation emitter.
17. The handle of aspect 16 or any other aspect herein wherein the second auxiliary reflector has a planar, concave, or convex reflective surface.
18. The handle of any one of aspects 2 to 17 or any other aspect herein wherein the ultraviolet radiation emitter comprises first and second radiation sources operable to emit ultraviolet radiation toward the concave surface of the reflector.
19. The handle of any one of aspects 2 to 3 or any other aspect herein wherein the ultraviolet radiation source is attached to the proximal end surface and wherein the ultraviolet radiation emitter comprises a second reflector having a convex surface located in a concavity of the reflector.
20. The handle of any one of aspects 2 to 19 or any other aspect herein wherein the ultraviolet radiation source and/or the ultraviolet radiation emitter comprises a principal optical axis which is aligned with a central axis of the grip.
21. The handle of any one of aspects 2 to 20 or any other aspect herein further comprising a base coupled to the grip at the proximal end of the grip, the base comprising attachment mechanisms for attachment onto a door.
22. The handle of aspect 21 or any other aspect herein wherein the base is configured to cause the longitudinal direction to be parallelly aligned with a leaf of the door when the handle is attached to the door.
23. The handle of aspect 21 or any other aspect herein wherein the base is configured to cause the longitudinal direction to be orthogonally aligned with a leaf of the door when the handle is attached to the door.
24. The handle of any one of aspects 2 to 23 or any other aspect herein wherein the concave surface of the reflector comprises a first section having a first curvature and a corresponding first focal point and a second segment having a second curvature and a corresponding second focal point.
25. The handle of aspect 24 or any other aspect herein wherein the first segment has a parabolic curvature and wherein the second segment has a hyperbolic curvature.
26. The handle of any of aspects 24 to 25 wherein the ultraviolet radiation source is spaced apart from the first and second focal points.
27. The handle of any one of aspects 1 to 26 or any other aspect herein wherein the an ultraviolet radiation source comprises a solid state UV emitter (e.g. a UV-LED).
28. A handle comprising:
   a grip having a body extending in a longitudinal direction between a proximal end surface located at a proximal end of the grip and a distal end surface located at an opposing distal end of the grip;
   an ultraviolet radiation emitter located at the proximal end of the grip and configured to direct ultraviolet radiation from the proximal end and in directions oriented toward the body, the ultraviolet radiation emitter comprising:
      an ultraviolet radiation source operable to emit the ultraviolet radiation; and
      a reflector having a concave surface disposed around the ultraviolet radiation source.
29. The handle of aspect 28 comprising any of the features of any of aspects 3 to 27.
30. A handle comprising:
   a grip having a body made of a UV transparent material extending in a longitudinal direction, the body extending between a proximal end surface located at a proximal end of the grip and a distal end surface located at an opposing distal end of the grip; and
an UV radiation source operable to emit UV radiation, the UV radiation source located at the proximal end of the grip and optically oriented to emit UV radiation through the proximal end surface and into an interior of the body of the grip,
wherein a shape and an index of refraction of the body are selected to cause the UV radiation in the interior of the body to undergo total internal reflection at an interface between the body and air as the UV radiation travels in the interior of the body.
31. The handle of aspect 30 or any other aspect herein wherein contact residue on an external surface of the body causes radiation in the interior of the body to be transmitted to an outside of the body and into the contact residue because of the difference between the index of refraction of the contact residue and air.
32. The handle of aspect 31 or any other aspect herein wherein the contact residue is residue from contact between human skin and the body.
33. The handle of aspect 31 or any other aspect herein wherein the contact residue comprises microorganisms.
34. The handle of any one of aspects 30 to 33 or any other aspect herein further comprising a second UV radiation source located at the distal end of the grip and optically oriented to emit UV radiation through the distal end surface and into the interior of the body.
35. The handle of any one of aspects 30 to 33 or any other aspect herein further comprising an end reflector located at the distal end of the grip, the end reflector configured to direct the UV radiation received from the UV radiation source toward the proximal end of the grip.
36. The handle of any one of aspects 1 to 35 or any other aspect herein further comprising:
   a sensor for detecting a presence of a user and/or a user's hand in contact with or in proximity to the handle; and
   control logic configured to turn the UV radiation source ON or OFF based on output from the sensor.
37. The handle of aspect 36 or any other aspect herein wherein the sensor comprises one or more of:
   a motion sensor for detecting motion associated with the presence of the user and/or the user's hand;
   a LiDAR sensor for detecting the presence of the user and/or the user's hand;
   a heat sensor for detecting heat associated with the presence of the user and/or the user's hand; and
   a proximity sensor for detecting the proximity of the user and/or the user's hand.
38. A method for disinfecting surfaces of a handle, the method comprising:
   providing a handle comprising a grip having a body extending in a longitudinal direction between a proximal end surface located at a proximal end of the grip and a distal end surface located at an opposing distal end of the grip;
   locating an ultraviolet radiation emitter comprising an ultraviolet radiation source at the proximal end of the grip and orienting the ultraviolet radiation emitter to direct ultraviolet radiation from the proximal end and in directions oriented toward the body; and
   disposing a concave reflector around the ultraviolet radiation source with the ultraviolet radiation source located at least partially in a cavity defined by the concave reflector.
39. The method of aspect 38 comprising any of the features, combinations of features and/or sub-combinations of features of any of aspects 1 to 37.
40. A method for disinfecting surfaces of a handle, the method comprising:
   providing a handle according to any one of aspects 1 to 37;
   sensing at least one of:
      a toggle off condition comprising at least one of: a presence of a user in a vicinity of the handle; a hand of a user in proximity with the handle; and a hand of the user in contact with handle; and
      a toggle on condition comprising at least one of: an absence of a user in a vicinity of the handle; an absence of a hand of a user in proximity with the handle; and an absence of hand of the user in contact with handle; and at least one of:
      causing the UV radiation source to turn ON (start emitting radiation) for at least a period of time based on sensing a toggle on condition; and
      causing the UV radiation source to turn OFF (stop emitting radiation) based sensing a toggle off condition.
41. The method of aspect 40 comprising any of the features, combinations of features and/or sub-combinations of features of any of aspects 1 to 39.
42. Apparatus having any new and inventive feature, combination of features, or sub-combination of features as described herein.
43. Methods having any new and inventive steps, acts, combination of steps and/or acts or sub-combination of steps and/or acts as described herein.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A handle comprising:
a grip having a body extending in a longitudinal direction between a proximal end surface located at a proximal end of the grip and a distal end surface located at an opposing distal end of the grip;
an ultraviolet radiation emitter located at the proximal end of the grip and configured to direct ultraviolet radiation from the proximal end and in directions oriented toward the body, the ultraviolet radiation emitter comprising:
an ultraviolet radiation source operable to emit the ultraviolet radiation; and
a housing disposed at least partially around the ultraviolet radiation source with the ultraviolet radiation source located at least partially in a cavity defined at least in part by the concave housing.

2. The handle of claim 1 wherein the housing comprises a reflector having a reflective surface disposed at least partially around the ultraviolet radiation source.

3. The handle of claim 2 wherein the ultraviolet radiation source is located at least partially in a reflector concavity defined at least in part by the surface of the reflector.

4. The handle of claim 2 wherein the ultraviolet radiation emitter is configured to direct ultraviolet radiation from the proximal end in directions oriented toward the body and having components oriented in the longitudinal direction and orthogonal to the longitudinal direction.

5. The handle of claim 4 wherein the concave surface is shaped for receiving the ultraviolet radiation from the ultraviolet radiation source and for directing the ultraviolet radiation received from the ultraviolet radiation source in the directions oriented toward the body and having components oriented in the longitudinal direction and orthogonal to the longitudinal direction.

6. The handle of claim 2 further comprising an auxiliary reflector located at the proximal end surface of the grip, the auxiliary reflector oriented to face toward the ultraviolet radiation source to reflect the ultraviolet radiation emitted by the ultraviolet radiation source toward the concave surface of the reflector.

7. The handle of claim 6 wherein the auxiliary reflector has a planar, concave, or convex reflective surface.

8. The handle of claim 2 further comprising an end cover located at the distal end of the grip.

9. The handle of claim 8 wherein the end cover comprises a reflective surface configured to direct the ultraviolet radiation received from the ultraviolet radiation emitter from the distal end in the directions oriented toward the body and having components oriented in the longitudinal direction and orthogonal to the longitudinal direction.

10. The handle of claim 9 wherein the end cover has a planar or concave reflective surface.

11. The handle of claim 8 wherein the end cover spans an area greater than the area of the distal end surface and wherein the end cover is attached to the distal end surface.

12. The handle of claim 2 further comprising a second ultraviolet radiation emitter located at the distal end of the grip and configured to direct ultraviolet radiation from the distal end and in directions oriented toward the body.

13. The handle of claim 12 wherein the second ultraviolet radiation emitter comprises:
a second ultraviolet radiation source operable to emit the ultraviolet radiation; and
a second reflector having a second concave surface disposed around the second ultraviolet radiation source.

14. The handle of claim 13 wherein the second ultraviolet radiation emitter is configured to direct ultraviolet radiation from the distal end in directions oriented toward the body and having components oriented in the longitudinal direction and orthogonal to the longitudinal direction.

15. The handle of claim 14 wherein the second concave surface is shaped for receiving the ultraviolet radiation from the second ultraviolet radiation source and for directing the ultraviolet radiation received from the second ultraviolet radiation source in the directions oriented toward the body and having components oriented in the longitudinal direction and orthogonal to the longitudinal direction.

16. The handle of claim 13 further comprising a second auxiliary reflector located at the distal end surface of the grip, the second auxiliary reflector oriented to face toward the ultraviolet radiation source of the second ultraviolet radiation emitter to reflect the ultraviolet radiation emitted by the ultraviolet radiation source toward the concave surface of the reflector of the second ultraviolet radiation emitter.

17. The handle of claim 16 wherein the second auxiliary reflector has a planar, concave, or convex reflective surface.

18. The handle of claim 2 wherein the ultraviolet radiation emitter comprises first and second radiation sources operable to emit ultraviolet radiation toward the concave surface of the reflector.

19. The handle of claim 2 wherein the ultraviolet radiation source is attached to the proximal end surface and wherein the ultraviolet radiation emitter comprises a second reflector having a convex surface located in a concavity of the reflector.

20. The handle of claim 2 wherein the ultraviolet radiation source and/or the ultraviolet radiation emitter comprises a principal optical axis which is aligned with a central axis of the grip.
